(19) United States
(12) Patent Application Publication
(10) Pub. No.: US 2024/0033368 A1
(43) Pub. Date: Feb. 1, 2024

(54) PREPARATION AND PURIFICATION METHOD OF ANTIBODY DRUG CONJUGATE INTERMEDIATE

(71) Applicant: REMEGEN CO., LTD., Yantai (CN)

(72) Inventors: Zhuanglin LI, Yantai (CN); Wei GUO, Yantai (CN); Quanzhou ZHAO, Yantai (CN); Zhonglin ZHANG, Yantai (CN)

(73) Assignee: REMEGEN CO., LTD., Yantai, SD (CN)

(21) Appl. No.: 18/375,175

(22) Filed: Sep. 29, 2023

Related U.S. Application Data
(63) Continuation of application No. PCT/CN2022/084238, filed on Mar. 31, 2022.

(30) Foreign Application Priority Data
Mar. 31, 2021 (CN) 202110350022.0

(51) Int. Cl. A61K 47/68

(52) U.S. Cl. CPC A61K 47/6809; A61K 47/68031; A61K 47/6889

(57) ABSTRACT

The disclosure relates to a preparation and purification method for an antibody drug conjugate intermediate, and more particularly relates to a preparation and purification method for a linker part and drug part conjugate in an antibody drug conjugate, which can not only effectively remove impurities from a target product and by-products in a reaction process, making the purity of the target product finally obtained up to 99% or above, but also realize stable mass production and well meet the quality standard requirements of clinical drugs, so as to provide a tremendous guarantee for drug safety and stable supply.

### CROSS-REFERENCE TO RELATED APPLICATIONS

This application claims priority to Chinese Application No. CN202110350022.0, filed Mar. 31, 2021, the disclosures of which are incorporated herein by reference in their entirety.

### TECHNICAL FIELD

The present invention relates to the field of pharmaceutical chemistry, and more particularly relates to a preparation and purification method for an antibody drug conjugate intermediate.

### BACKGROUND

Antibody drug conjugates (ADCs) are a type of antineoplastic drugs, which includes three components: an antibody part (Antibody), a linker part (Linker) and a toxin part (Drug). The antibody part and the toxin part are linked by the linker part, and the mechanism of action is to use the targeting of antibodies for target transport of drugs to target cells (such as tumor cells) and release toxins to kill tumor cells. For the time being, the most common method for synthesizing antibody drug conjugates is to covalently link the linker part and the toxin part in the liquid phase to form a linker-toxin conjugate, and then conduct sulfhydryl or amino conjugating with an antibody to form an antibody drug conjugate. For example, the linker-toxin structure on the antibody of several listed ADC drugs is Mc-Val-Cit-PAB-MMAE. However, because MMAE is very expensive, the cost of producing a batch of Mc-Val-Cit-PAB-MMAE is as high as millions of RMB (MMAE feeding is at the level of 100 grams). Therefore, a synthesis and purification method with controllable quality, high yield and high purity is required to produce such ADC intermediates, so as to stably supply ADC drugs in batches.

The Publication No. CN 108853514A discloses a preparation and purification method for an antibody drug conjugate intermediate (Mc-Val-Cit-PAB-MMAE) on Page 14 of the Description:

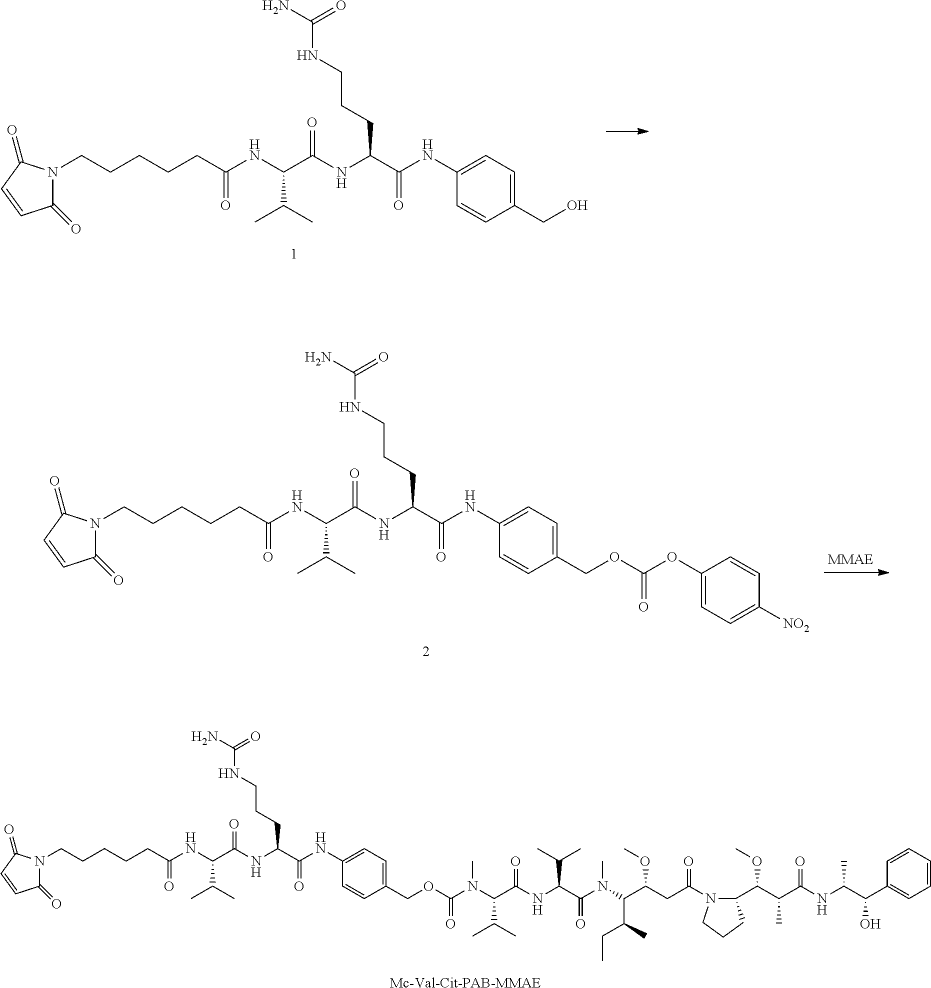

The method is mainly divided into two steps. The first step is to prepare Mc-VC-PAB-PNP with the compound Mc-VC-PABA, and the second step is to prepare Mc-Val-Cit-PAB-MMAE with Mc-VC-PAB-PNP and MMAE. The product Mc-VC-PAB-PNP in the first step is crystallized with petroleum ether and ethyl acetate, and the product is used in the next step without purification, which undoubtedly brings more impurities to the subsequent reaction; and the product purification process in the second step only involves the preparation and purification by HPLC, so the prepared compound often has high impurity content. The Chinese patents with Publication No, CN106999605A and CN108743968A also disclose a preparation method for Mc-Val-C t-PAB MMAE on Page 47 of the Description and Page 4 of the Description, but the subsequent product purification methods are the preparation and purification by HPLC, which cannot effectively remove impurities from the product.

### SUMMARY OF THE INVENTION

In order to solve the above-identified problems, the disclosure provides new methods of generating purified linker-toxin conjugates that can be used as intermediates to synthesize ADCs. The disclosure also provides purified Mc-Val-Cit-PAB-D of the compound of Formula (H, or a salt thereof, where D represents the linked toxin part.

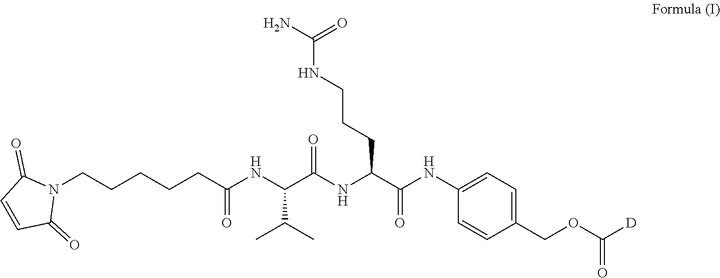 [SMILES: [2H]C(=O)OCc1ccc(NC(=O)[C@H](CCCNC(N)=O)NC(=O)[C@@H](NC(=O)CCCCCN2C(=O)C=CC2=O)C(C)C)cc1]

It will be understood that the methods disclosed herein can also be used to make racemic versions, diastereomers or enantiomers of the compound of Formula (I), or salts thereof.

It has been surprisingly discovered that highly pure compound of Formula (I), or a salt thereof, can be generated reacting Compound 2 and a toxin (D) (e.g., MMAE) in the presence of a triazole-based compound, as depicted below:

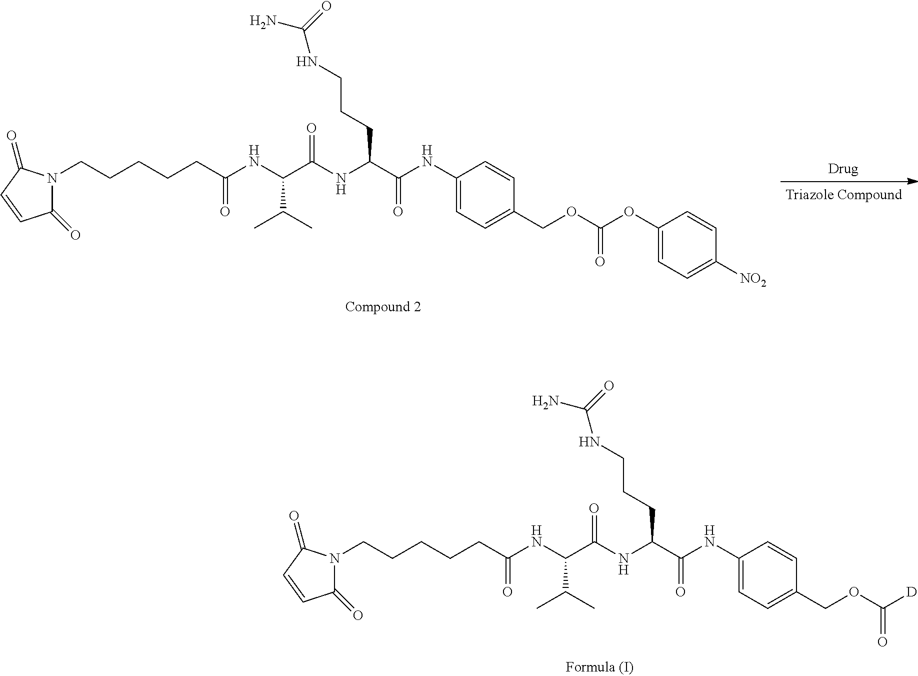 [SMILES: CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1 ; [2H]C(=O)OCc1ccc(NC(=O)[C@H](CCCNC(N)=O)NC(=O)[C@@H](NC(=O)CCCCCN2C(=O)C=CC2=O)C(C)C)cc1]

In some embodiments, the triazole-based compound is 1-hydroxybenzotriazole, 1-hydroxy-7-azobenzotriazole, ethyl 1-hydroxyl-1H-1,2,3-triazole-4-carboxylate, or combinations thereof. In particular embodiments, the triazole-based compound is 1-hydroxybenzotriazole.

In some embodiments, the reaction depicted above is run in the presence of one or more bases. For instance, the reactions can be run in the presence of one or more organic bases. In particular embodiments, the reaction is one in the presence of two organic bases of different alkalinity. It has been found that using two different organic bases with differing alkalinity further reduces the amount of impurities generated in the process. In one embodiment, at least one of the organic bases is N,N-Diisopropylethylamine. In another embodiment, at least one of the organic bases is pyridine. In another embodiment, the two organic bases are N,N-Diisopropylethylamine and pyridine.

The disclosure further provides methods of synthesizing Compound 2. In one embodiment, Compound 2 is prepared by reacting Compound 1 with bis-(4-nitrobenzene) in the presence of an organic base, referred to herein as Organic Base 1. The reaction is depicted below:

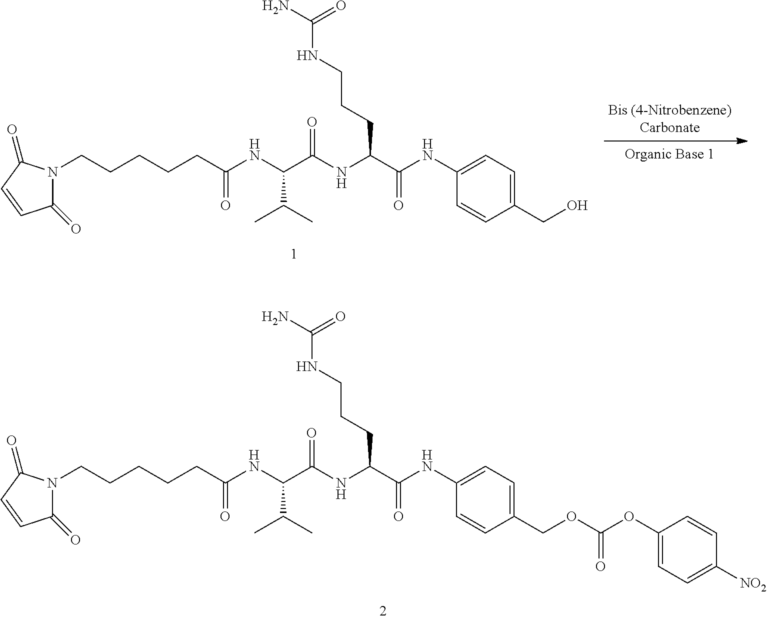 [SMILES: CC(C)[C@H](NC(=O)CCCCCN1C(=O)C=CC1=O)C(=O)N[C@@H](CCCNC(N)=O)C(=O)Nc1ccc(CO)cc1 ; CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1]

In some embodiments, the toxin part D is an auristatin cytotoxic agent, an Anthramycin cytotoxic agent, an anthracycline cytotoxic agent or a puromycin cytotoxic agent, wherein the auristatin cytotoxic agent includes MMAE, MMAF, MMAD or derivatives thereof: the Anthramycin cytotoxic agent includes anthramycin or derivatives thereof; the anthracycline cytotoxic agent includes daunorubicin, adriamycin, epirubicin, idarubicin, valrubicin, mitoxantrone or derivatives thereof: the puromycin cytotoxic agent includes puromycin or derivatives thereof;

In particular embodiments, the toxin (D) is MMAE. In such embodiments, the purified linker-toxin conjugates produced in accordance with the disclosure is Mc-Val-Cit-PAB-MMAE, which has the following chemical structure:

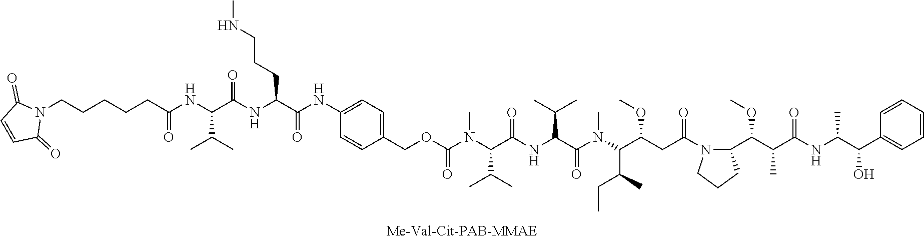

In some embodiments, Mc-Val-Cit-PAB-D is produced using the following synthetic route:

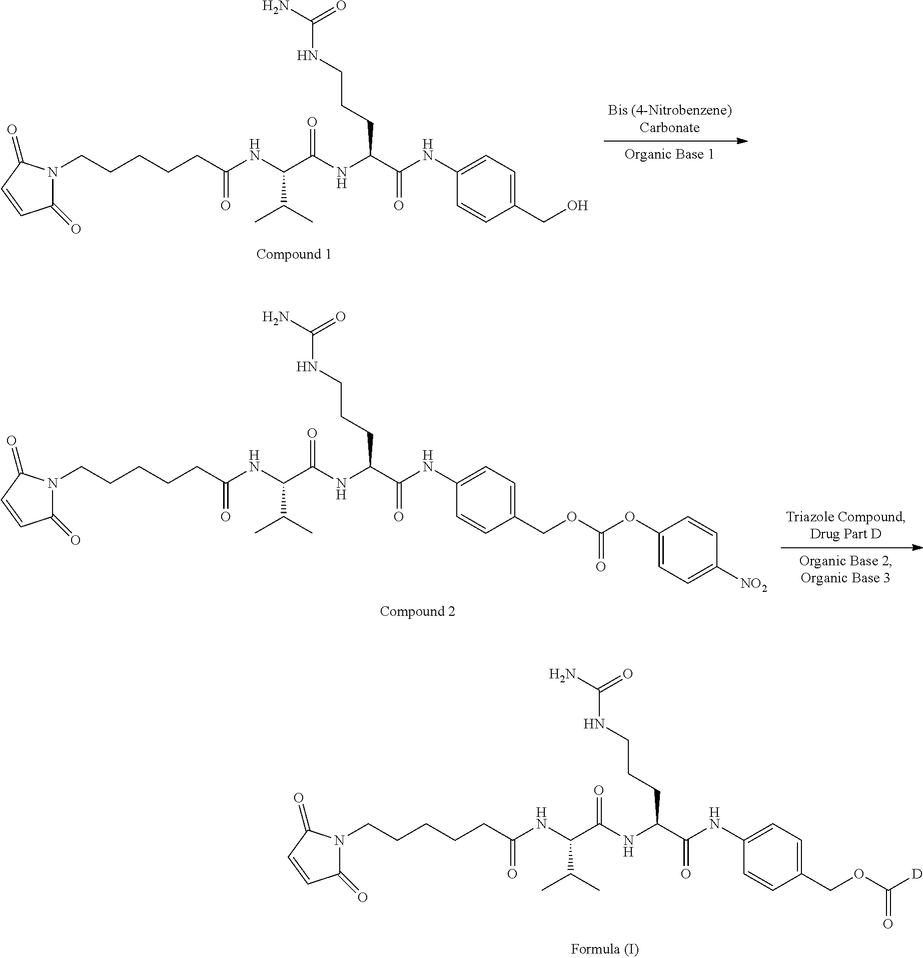 [SMILES: CC(C)[C@H](NC(=O)CCCCCN1C(=O)C=CC1=O)C(=O)N[C@@H](CCCNC(N)=O)C(=O)Nc1ccc(CO)cc1 ; CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1 ; [2H]C(=O)OCc1ccc(NC(=O)[C@H](CCCNC(N)=O)NC(=O)[C@@H](NC(=O)CCCCCN2C(=O)C=CC2=O)C(C)C)cc1]

In particular embodiments, D is MMAE.

In some embodiments, the Mc-Val-Cit-PAB-D (e.g., Mc-Val-Cit-PAB-MMAE) produced by methods described herein can have a purity of greater than 95% In some embodiments, the Mc-Val-Cit-PAB-D (e.g., Mc-Val-Cit-PAB-MMAE) produced by methods described herein can have a purity of greater than 96%. In some embodiments, the Mc-Val-Cit-PAB-D (e.g., Mc-Val-Cit-PAB-MMAE) produced by methods described herein can have a purity of greater than 97%. In some embodiments, the Mc-Val-Cit-PAB-D (e.g., Mc-Val-Cit-PAB-MMAE) produced by methods described herein can have a purity of greater than 98%. In some embodiments, the Mc-Val-Cit-PAB-D (e.g., Mc-Val-Cit-PAB-MMAE) produced by methods described herein can have a purity of greater than 99%. In some embodiments, the Mc-Val-Cit-PAB-D (e.g., Mc-Val-Cit-PAB-MMAE) produced by methods described herein can have a purity of greater than 99.5%. In some embodiments, the Mc-Val-Cit-PAB-D (e.g., Mc-Val-Cit-PAB-MMAE) produced by methods described herein can have a purity of greater than 99.8%. %. In other embodiments, the Mc-Val-Cit-PAB-D (e g., Mc-Val-Cit-PAB-MMAE) produced by methods described herein can have a purity of from about 95% to about 99.5%. In other embodiments, the Mc-Val-Cit-PAB-D (e.g., Mc-Val-Cit-PAB-MMAE) produced by methods described herein can have a purity of from about 97% to about 99.5%. In other embodiments, the Mc-Val-Cit-PAB-D (e.g., Mc-Val-Cit-PAB-MMAE) produced by methods described herein can have a purity of from about 98% to about 99.8%. In other embodiments, the Mc-Val-Cit-PAB-D (e.g., Mc-Val-Cit-PAB-MMAE) produced by methods described herein can have a purity of from about 95% to about 98%.

The disclosure also provides highly pure ADCs produced using purified linker-toxin conjugates as prepared herein. In some embodiments, the ADCs produced in accordance with the disclosure can have a purity of greater than 95%. In other embodiments, the ADCs produced in accordance with the disclosure can have a purity of greater than 96%. In other embodiments, the ADCs produced in accordance with the disclosure can have a purity of greater than 97%. In other embodiments, the ADCs produced in accordance with the disclosure can have a purity of greater than 98%. In other embodiments, the ADCs produced in accordance with the disclosure can have a purity of greater than 99%. In other embodiments, the ADCs produced in accordance with the disclosure can have a purity of greater than 99.5%. In other embodiments, the ADCs produced in accordance with the disclosure can have a purity of greater than 99.8%. In other embodiments, the ADCs produced in accordance with the disclosure can have a purity of from about 95% to about 99.5%.

In one embodiment, the disclosed method includes the following steps:
A. dissolving Compound 1 in an appropriate amount of Solvent 1, and successively adding bis(4-nitrobenzene) carbonate and organic base, wherein the number of moles of the added bis(4-nitrobenzene) carbonate and the number of moles of the added organic base are greater than that of Compound 1;
B. obtaining the filtrate by suction filtration after appropriate reaction time;
C. successively adding a sufficient amount of ethyl acetate and n-hexane to the filtrate obtained in Step B, stirring for an appropriate time after n-hexane has been dropwise added, and obtaining the filter cake by suction filtration.
D. successively washing the filter cake obtained in Step C with an appropriate amount of ethyl acetate and n-hexane, and obtaining the filter cake by suction filtration;
E. dissolving the filter cake obtained in Step D in a mixed solution of acetic acid and methanol, adding an appropriate amount of purified water, stirring for an appropriate time after the purified water is added, and obtaining the filter cake by suction filtration:
F. successively washing the filter cake obtained in Step E with an appropriate amount of purified water, methanol, ethyl acetate and n-hexane, and obtaining Compound 2 (MC-Val-Cit-PAB-PNP) after suction filtration and drying;
G. dissolving Compound 2 and a triazole-based compound in an appropriate amount of Solvent 2 to form Solution X, dissolving the conjugated toxin part D in Solvent 3 to form Solution Y, adding Solution Y into Solution X, and mixing evenly to form Solution Z;
H, adding an appropriate amount of organic base to Solution Z;
I. obtaining the filtrate by suction filtration after appropriate reaction time:
J. successively adding an appropriate amount of ethyl acetate and n-hexane to the filtrate in Step I, stirring for an appropriate time, and obtaining the filter cake by suction filtration;
K. successively washing the filter cake obtained in Step J with ethyl acetate and n-hexane, and obtaining the filter cake by suction filtration;
L. dissolving the filter cake obtained in Step K in an appropriate amount of methanol solution, preparing and purifying by high performance liquid chromatography, and collecting a preparation solution;
M. concentrating the preparation solution obtained in Step L under reduced pressure;
N. dissolving the concentrate obtained under reduced pressure in Step M with an appropriate amount of methanol, and then concentrating under reduced pressure again;
O. vacuum drying the concentrate obtained under reduced pressure in Step N to obtain the purified compound as shown in Formula (I);
wherein:

In some embodiments, solvent 1 in Step A. Solvent 2 and Solvent 3 in Step G are polar solvents; preferably, Solvent 1, Solvent 2 and Solvent 3 are each independently selected from one or more of DMF, DMA and NMP; and more preferably, Solvent 1, Solvent 2 and Solvent 3 are DMF:

The organic base in Step A and the organic base in Step H are selected from one or more of N,N-Diisopropylethylamine, triethylamine and pyridine; preferably, the organic bases are each independently one or two of N,N-Diisopropylethylamine and pyridine. Preferably, the organic base in Step A is N,N-Diisopropylethylamine, and there are two types of organic bases, N,N-Diisopropylethylamine and pyridine, in Step H.

Further, in some embodiments, the molar ratio of Compound 1 in Step A to bis(4-nitrobenzene) carbonate is about 1:1.8, and the molar ratio of Compound 1 to organic base 1 is about 1:1.2.

Further, in some embodiments, the molar ratio of Compound 1 in Step A to bis(4-nitrobenzene) carbonate is 1:1.5-2, and the molar ratio of Compound 1 to organic base is 1:1-1.5. Preferably, the molar ratio of Compound 1 in Step A to bis(4-nitrobenzene) carbonate is 1-1.6-1.9 or 1-1.7-1.8; and the molar ratio of Compound 1 to organic base is 1:1.1-1.4 or 1:1.2-1.3. In some specific embodiments, the molar ratio of Compound 1 in Step A to bis(4-nitrobenzene) carbonate is 1:1.8, and the molar ratio of Compound 1 to organic base is 1:1.2.

Further, in some embodiments, the weight volume ratio (g/ml) of Compound 1 to ethyl acetate in Step C is about 1:30.0, and the weight volume ratio (g/ml) of Compound 1 to n-hexane in Step C is about 1:60.0.

Further, in some embodiments, the weight volume ratio (g/ml) of Compound 1 to ethyl acetate in Step C is 1:25-35, 1:27-33, 1:28-32, or 1:29-31; and the weight volume ratio (g/ml) of Compound 1 to n-hexane in Step C is 1:55-65, 1:57-63, 1:58-62, or 1:59-61. In some specific embodiments, the weight volume ratio (g/ml) of Compound 1 to ethyl acetate in Step C is 1:30, and the weight volume ratio (g/ml) of Compound 1 to n-hexane in Step C is 1:60.

Further, in some embodiments, the weight volume ratio (g/ml) of Compound 1 to acetic acid in Step E is about 1:7.0, the weight volume ratio (g/ml) of Compound 1 to methanol in Step E is about 1:1.0, and the weight volume ratio (g/ml) of Compound 1 to purified water in Step E is about 1:20.0.

Further, in some embodiments, the weight volume ratio (g/ml) of Compound 1 to acetic acid in Step E is about 1:6-8, the weight volume ratio (g/ml) of Compound 1 to methanol in Step E is 1:0.5-1.5, and the weight volume ratio (g/ml) of Compound 1 to purified water in Step E is 1:15-25. Preferably, the weight volume ratio (g/ml) of Compound 1 to acetic acid in Step E is 1:6.5-7.5 or 1.6.8-7.3; the weight volume ratio (g/ml) of Compound 1 to methanol in Step E is 1:0.7-1.3 or 1:0.9-1.1; and the weight volume ratio (g/ml) of Compound 1 to purified water in Step E is 1:17-23 or 1:19-21. In some specific embodiments, the weight volume ratio (g/ml) of Compound 1 to acetic acid in Step E is 1:7.0, the weight volume ratio (g/ml) of Compound 1 to methanol in Step E is 1:1.0; and the weight volume ratio (g/ml) of Compound 1 to purified water in Step E is 1:20.0.

Further, in some embodiments, the molar ratio of Compound 2 to triazole-based compound in Step G is about 1:1, and the molar ratio of Compound 2 to toxin part D is about 1:1.

Further, in some embodiments, the molar ratio of Compound 2 to triazole-based compound in Step G is 1:0.8-1.2, and the molar ratio of Compound 2 to toxin part D is 1:0.8-1.2. In some embodiments, the molar ratio of Compound 2 to triazole-based compound in Step G is 1:0.9-1.1, and the molar ratio of Compound 2 to toxin part D is 1:0.9-1.1. Preferably, the molar ratio of Compound 2 to triazole-based compound in Step G is 1:0.85-1.05, and the molar ratio of Compound 2 to toxin part D is 1:0.95-1.05. In some specific embodiments, the molar ratio of Compound 2 to triazole-based compound in Step G is 1:1, and the molar ratio of Compound 2 to toxin part D is 1:1.

Further, as set forth above, in some embodiments there are two types of organic bases, N,N-Diisopropylethylamine and pyridine, in Step H. In some embodiments, the molar ratio of Compound 2 in Step G to the organic base N,N-Diisopropylethylamine added in Step H is about 1:1, and the molar ratio of Compound 2 in Step G to the organic base pyridine added in Step H is about 1:20.5. In other embodiments, the molar ratio of Compound 2 in Step G to the organic base N,N-Diisopropylethylamine added in Step H is 1:0.8-1.2, and more preferably, the molar ratio is 1:0.9-1.1 or 1:0.95-1.05; the molar ratio of Compound 2 in Step G to the organic base pyridine added in Step H is 1:19-25, and more preferably, the molar ratio is 1:19.5-23, 1:19.5-21.5 or 1:20-21. In some specific embodiments, the molar ratio of Compound 2 in Step G to organic base 2 is 1:1, and the molar ratio of Compound 2 to organic base 3 is 1:20.5.

Further, in some embodiments, the volume of the ethyl acetate added in Step J is 3.5-4.5 times the volume of the filtrate, and the volume of the n-hexane added is 7-9 times the volume of the filtrate. Preferably, the volume of the ethyl acetate added in Step J is 3.7-4.3 times the volume of the filtrate, and the volume of the n-hexane added is 7.5-8.5 times the volume of the filtrate. In some specific embodiments, the volume of the ethyl acetate added in Step J is 4 times the volume of the filtrate, and the volume of the n-hexane added is 8 times the volume of the filtrate.

Further, in some embodiments, the preparation conditions of high performance liquid chromatography in Step L are as follows: mobile phase A is an acetic acid aqueous solution with pH=4.0-5.0, mobile phase B is acetonitrile, mobile phase A:B=60:40(V/V), isogradient is used for preparation and purification.

Further, in particular embodiments, the structure of the antibody drug conjugate intermediate is shown in Formula (I-11):

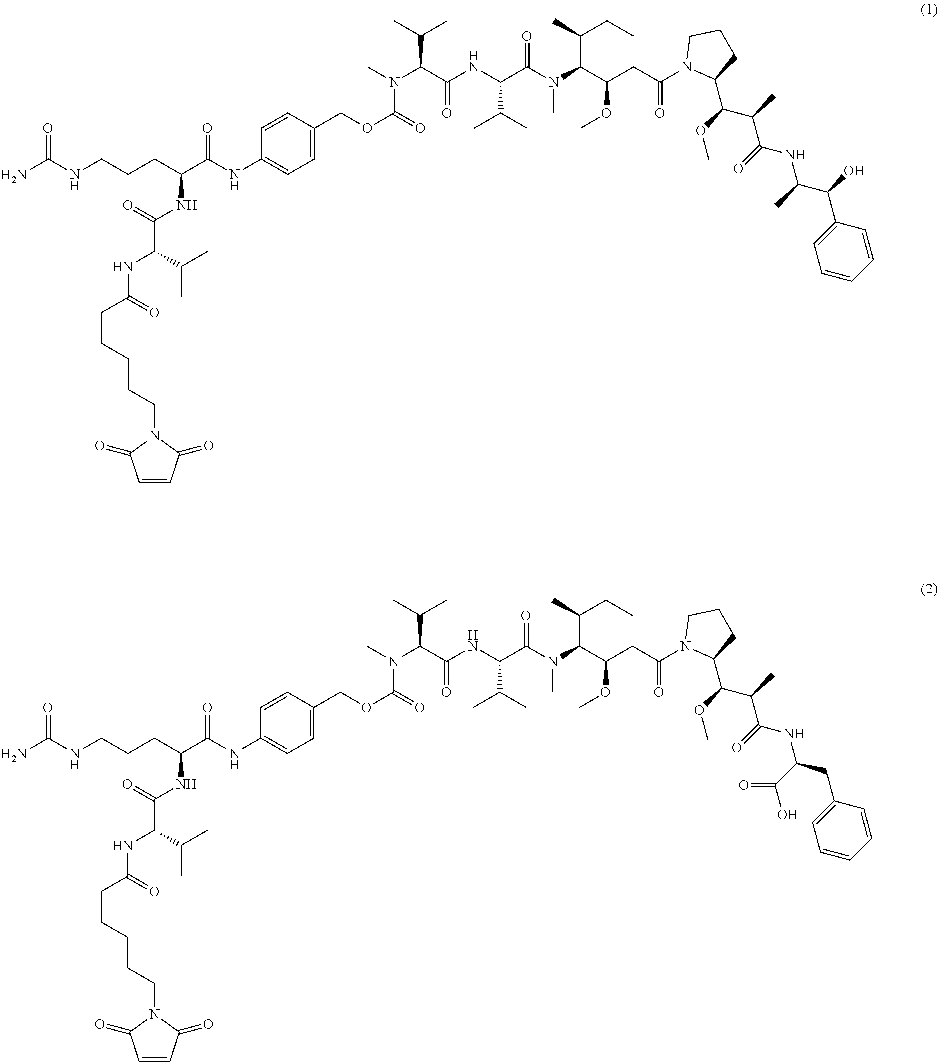 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C ; CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C]

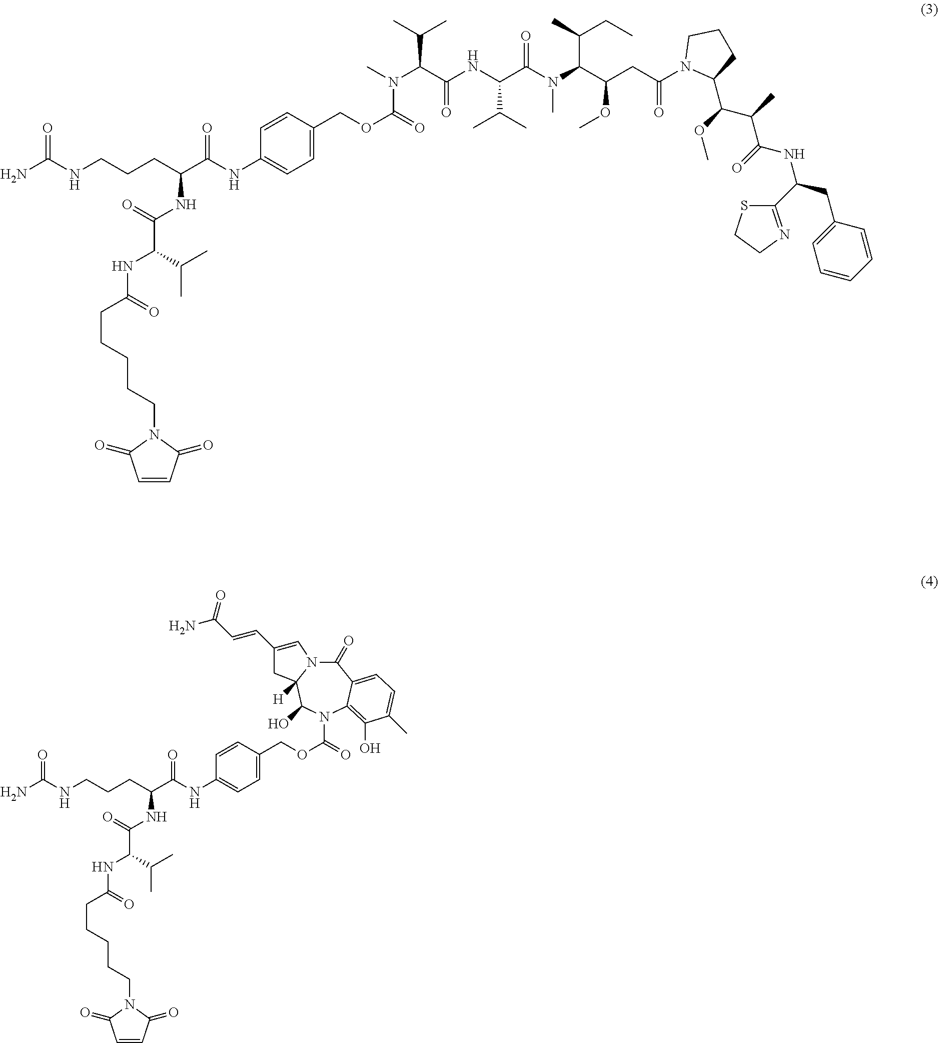 [SMILES: CC[C@H](C)[C@@H]([C@@H](CC(=O)N1CCC[C@H]1[C@H](OC)[C@@H](C)C(=O)N[C@@H](Cc1ccccc1)C1=NCCS1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C ; Cc1ccc2c(c1O)N(C(=O)OCc1ccc(NC(=O)[C@H](CCCNC(N)=O)NC(=O)[C@@H](NC(=O)CCCCCN3C(=O)C=CC3=O)C(C)C)cc1)[C@@H](O)[C@@H]1CC(/C=C/C(N)=O)=CN1C2=O]

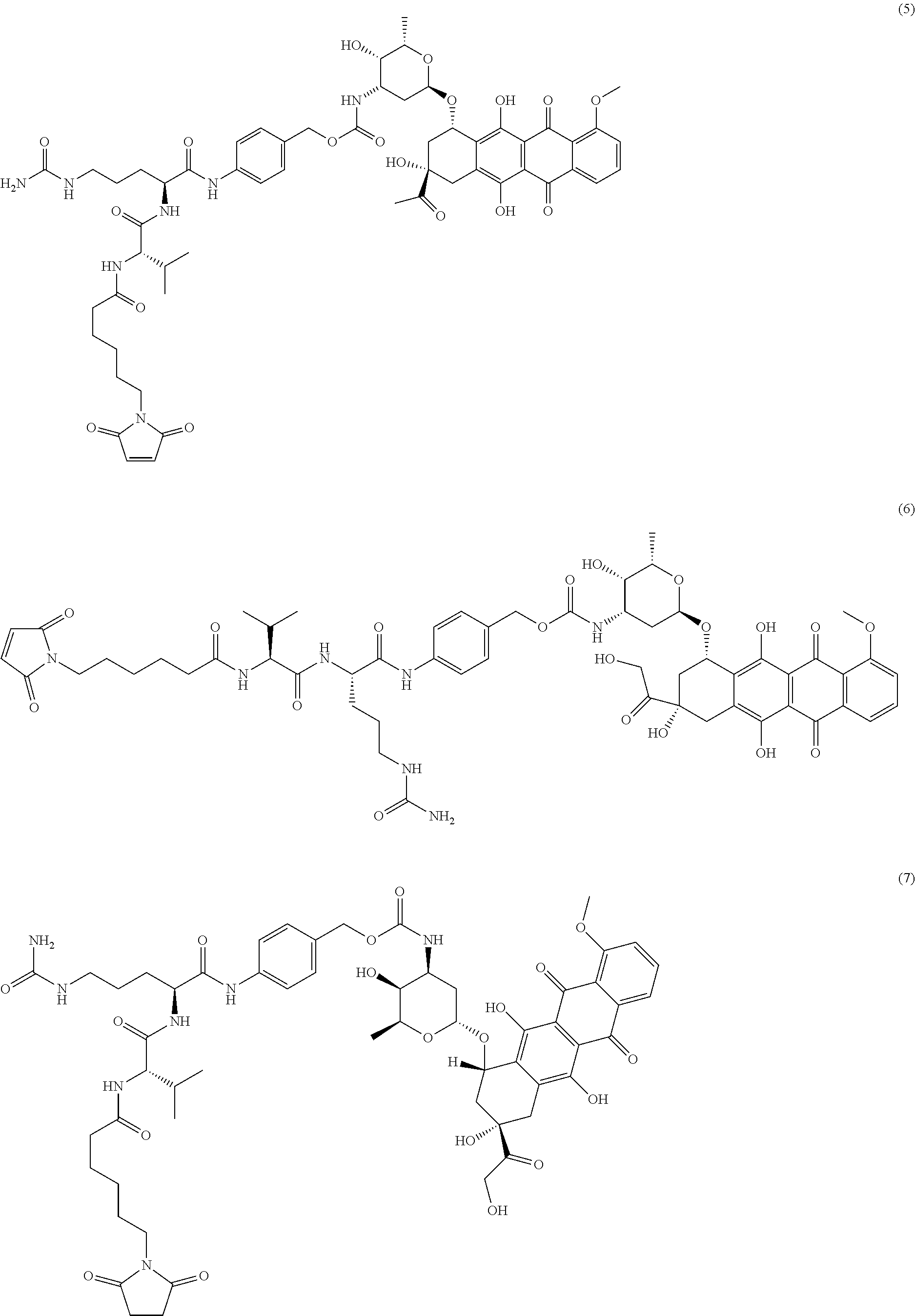

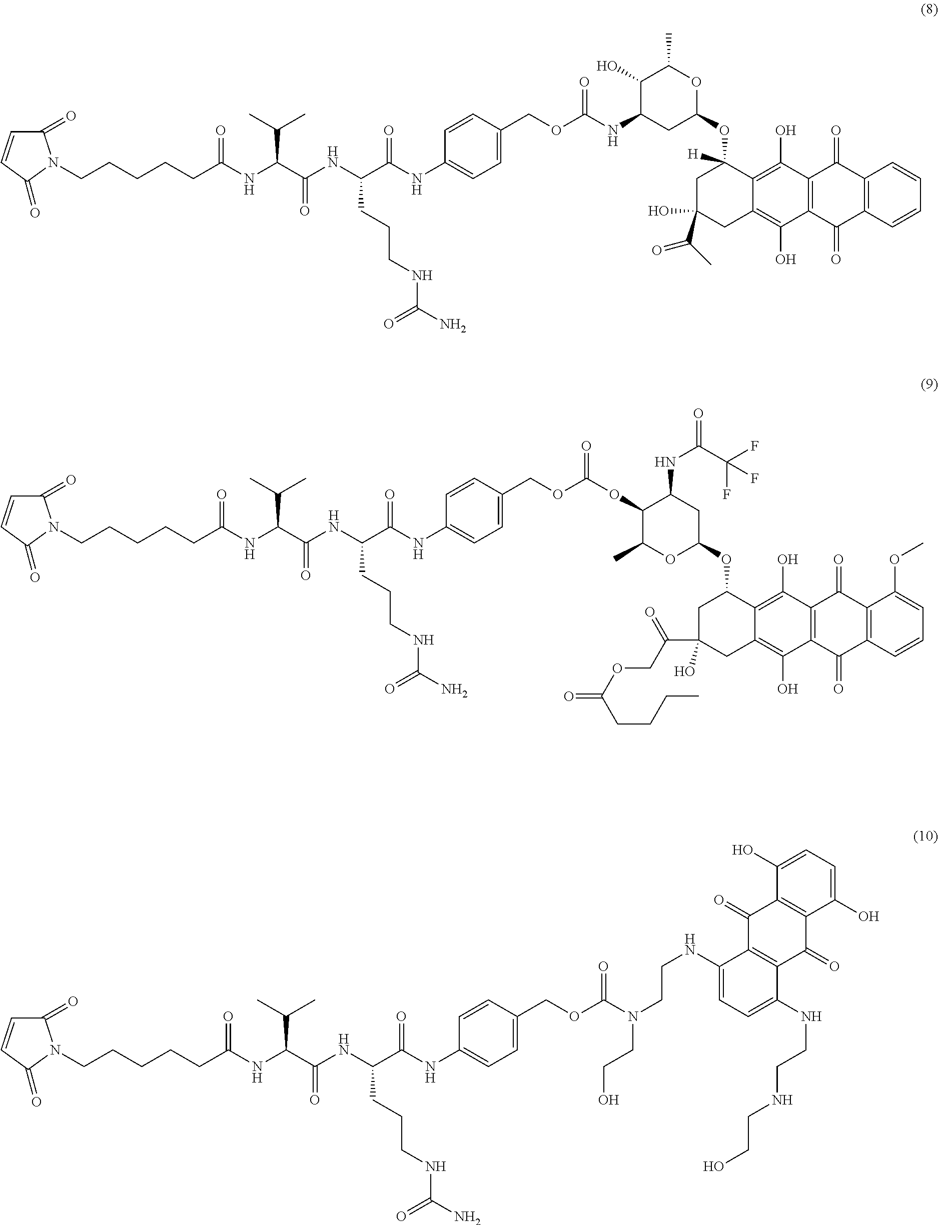

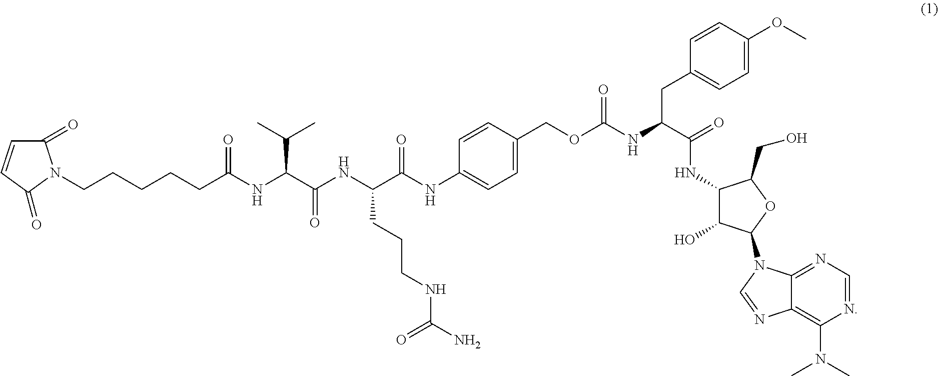 [SMILES: COc1ccc(C[C@H](NC(=O)OCc2ccc(NC(=O)[C@H](CCCNC(N)=O)NC(=O)[C@@H](NC(=O)CCCCCN3C(=O)C=CC3=O)C(C)C)cc2)C(=O)N[C@H]2[C@@H](O)[C@H](n3cnc4c(N(C)C)ncnc43)O[C@@H]2CO)cc1]

Further, in some embodiments, the temperature in Step A is controlled within the range of −5-5° C.

Further, in some embodiments, the temperature in Step B is controlled within the range of 25-30° C.

Further, in some embodiments, Step D can be repeated 1-5 times.

Further, in some embodiments, the washing times in Step F are 1-5 times.

Further, in some embodiments, the drying temperature in Step F is 25-30° C.

Further, in some embodiments, the triazole-based compound in Step G are one or more of 1-hydroxybenzotriazole, 1-hydroxy-7-azobenzotriazole and ethyl 1-hydroxyl-1H-1,2,3-triazole-4-carboxylate, preferably 1-hydroxybenzotriazole.

Further, in some embodiments, the temperature in Step G is controlled within the range of −5-5° C.

Further, in some embodiments, the temperature in Step H is controlled within the range of −5-5° C.

Further, in some embodiments, the reaction temperature in Step is 25-30° C.

Further, in some embodiments, the washing times in Step K are 1-5 times.

Further, in some embodiments, the temperature for concentration under reduced pressure in Step M is 25-35° C.

Further, in some embodiments, Step M is to concentrate the preparation solution obtained in Step L under reduced pressure to a foamed solid state.

Further, in some embodiments, the temperature for concentration under reduced pressure in Step N is 25-35° C.

Further, in some embodiments, Step N is to dissolve the concentrate under reduced pressure in Step M with an appropriate amount of methanol, and then concentrate under reduced pressure again to a foamed solid state.

Further, in some embodiments, Step N can be repeated 1-5 times.

Further, in some embodiments, Step A, Step B, Step G. Step H and Step I are all carried out under the protection of nitrogen.

The preparation and purification method for an antibody drug conjugate intermediate provided by the present invention can effectively remove the impurities from the target product and by-products in the reaction process, making the purity of the final target product obtained extremely high (e.g., 99% or above), and can realize stable mass production, and well meet the quality standard requirements of clinical drugs, so as to provide a tremendous guarantee for the stable mass production of ADC drugs.

### BRIEF DESCRIPTION OF THE DRAWINGS

FIG. 1 is a chromatogram of purified MC-Val-Cit-PAB-PNP.

FIG. 2 is a chromatogram of purified MC-VC-PAB-MMAE

### DETAILED DESCRIPTION

### Definitions

Unless otherwise defined, all technical terms used in the present invention have the same meaning as understood by a person of ordinary skill in the art.

The term “Antibody Drug Conjugate” used in the present invention refers to a compound whose antibody/functional fragment for antibody, linker and toxin part are linked together by chemical reaction, and is usually structurally consists of three parts: an antibody or antibody-based ligand, a toxin part and a linker conjugating the antibody or antibody-based ligand and a drug. Currently, an antibody drug conjugate is usually prepared in two steps: the first step is to form a “Linker-Drug” conjugate by chemical reaction between the linker and the toxin part, and the second step is to covalently couple the linker part in the “Linker-Drug” conjugate with the antibody/functional fragment for antibody by sulfhydryl or amino groups. The term “Antibody Drug Conjugate Intermediate” used in the present invention refers to the above “Linker-Drug” conjugate.

The terms “Linker” and “Linker Part” used in the present invention refer to the part that links the antibody to the drug in the antibody drug conjugating, which may be cleavable or non-cleavable. The cleavable linker (i.e., a breakable linker or a biodegradable linker) can be broken in or on a target cell to release drugs. In some embodiments, the linker of the present invention is selected from cleavable linkers, such as disulfide-based linkers (which are selectively broken in tumor cells with higher sulfhydryl concentration), peptide linkers (which are cut by enzymes in tumor cells) and hydrazone linkers. In other embodiments, the linker of the present invention is selected from non-cleavable linkers (i.e., non-breakable linkers), such as thioether linkers. In another embodiment, the linker of the present invention is a combination of breakable linkers and non-breakable linkers.

The terms “Drug” and “Toxin part” used in the present invention generally refer to any compound with desired biological activity and having reactive functional groups to prepare the conjugate of the present invention. The desired biological activities include diagnosing, curing, alleviating, treating, and preventing diseases in humans or other animals. With the continuous discovery and development of new drugs, these new drugs should also be included in the drugs described in the present invention. Specifically, the drugs include but are not limited to cytotoxic drugs, cell differentiation factors, stem cell nutrition factors, steroid-based drugs, drugs for treating autoimmune diseases, anti-inflammatory drugs, or drugs for infectious diseases. More specifically, the drugs include but are not limited to tubulin inhibitors or DNA and RNA damaging agents.

### EXAMPLES

The technical solutions of the present invention will be further described in non-limiting detail below in conjunction with the specific implementations. It should be noted that the following examples are only to illustrate the technical concept and characteristics of the present invention, and to enable those skilled in the art to understand the content of the present invention and implement it accordingly and cannot limit the protection scope of the present invention. All equivalent changes or modifications made according to the spirit essence of the present invention should be included in the protection scope of the present invention.

### Example 1 The Preparation and Purification for MC-Val-Cit-PAB-PNP

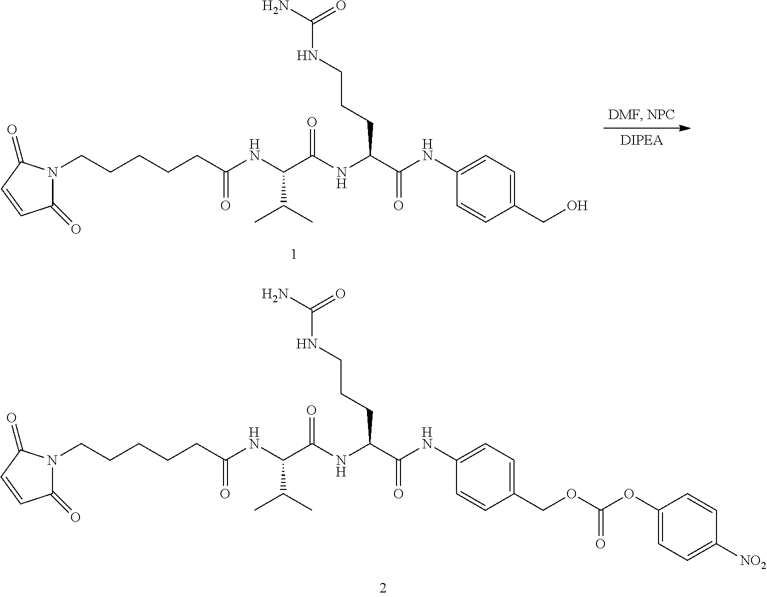 [SMILES: CC(C)[C@H](NC(=O)CCCCCN1C(=O)C=CC1=O)C(=O)N[C@@H](CCCNC(N)=O)C(=O)Nc1ccc(CO)cc1 ; CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1]

A clean and dry 3 L reaction flask was taken, into which 130.00 g of Compound 1 (i.e., MC-Val-Cit-PAB-OH) (227.01 mmol) and 1300 ml of DMF were added.

Stirring was carried out under the protection of nitrogen to disperse solids evenly, and the internal temperature was kept within the range of −2-2° C.

In the adding process, the internal temperature was controlled within the range of 0-5° C., and 124.02 g of bis(4-nitrobenzene) carbonate (407.68 mmol) was added.

In the dropwise adding process, the internal temperature was controlled within the range of 0-5° C., 35.03 g of N,N-Diisopropylethylamine (271.03 mmol) was dropwise added, the reaction solution turned brown in the dropwise adding process, and the temperature was raised after dropwise adding.

When the temperature was raised to 25° C., timing was started, the internal temperature was controlled to 25-30° C., samples were taken after reaction for 2 h, then samples were taken every 0.5 h, and in-process control detection was carried out. When the residual of Compound 1 was <1.0%, the reaction was terminated.

The above reaction solution was subjected to suction filtration, the reaction solution was taken and transferred to a 20 L stainless steel barrel, under mechanical stirring (100-300 rpm), 3900 ml of ethyl acetate (V_{ethyl acetate}/W_{compound 1}=30.0) was dropwise added, then 7800 ml of n-hexane (Vₙ₋ₕₑₓₐₙₑ/W_{compound 1}=60.0) was dropwise added, stirring was continued for 5±1 minutes after dropwise adding, and suction filtration was carried out with a circulating water multipurpose vacuum pump to obtain the filter cake, which was the crude product of Compound 2 (MC-Val-Cit-PAB-PNP).

5220 ml of ethyl acetate (V_{ethyl acetate}/W_{compound 1}=40.0) was taken and divided into 3 parts evenly. First the vacuum was removed, then a part of ethyl acetate was added to soak and wash the filter cake for 3-5 minutes, the filter cake was ground while soaking, then the vacuum was connected, the ethyl acetate was drawn out, and this operation was repeated twice.

5220 ml of n-hexane (Vₙ₋ₕₑₓₐₙₑ/W_{compound 1}=40.0) was taken and divided into 3 parts evenly. First the vacuum was removed, then a part of n-hexane was added to soak and wash the filter cake for 3-5 minutes, the filter cake was ground while soaking, then the vacuum was connected, the n-hexane was drawn out, and this operation was repeated twice; and the filter cake was subjected to suction filtration with a circulating water multipurpose vacuum pump until the product was a powder solid.

The obtained solid powder was transferred to a 10 L stainless steel barrel and dissolved with a mixed solution of 910 ml of acetic acid (V_{acetic acid}/W_{compound 1}=70.0) and 130 ml of methanol (Vₘₑₜₕₐₙₒₗ/W_{compound 1}=1.0); and under mechanical stirring (100-300 rpm), 2600 ml of purified water (V_{purified water}/W_{compound 1}=20.0) was dropwise added within 30±10 minutes. Stirring was continued for about 10 minutes after dropwise adding, and then suction filtration was carried out with a circulating water multipurpose vacuum pump to obtain the filter cake.

The filter cake was washed successively with purified water, methanol, ethyl acetate and ii-hexane. The specific washing method was as follows:

Washing with purified water, 2600 ml of purified water (V_{purified water}/W_{compound 1} i=20.0) was taken and divided into 2 parts evenly. First the vacuum was removed, purified water was added to soak and wash the filter cake for 3-5 minutes, the filter cake was ground while soaking, then the vacuum was connected, and the purified water was drawn out, and then the filter cake was rinsed with purified water under vacuum.

Washing with methanol: Under vacuum, 1300 ml of methanol (Vₘₑₜₕₐₙₒₗ/W_{compound 1}=10.0) was taken to evenly wash the filter cake.

Washing with ethyl acetate: 2610 ml of ethyl acetate (Vₘₑₜₕₐₙₒₗ/W_{compound 1}=20.0) was taken and divided into 3 parts evenly. First the vacuum was removed, then a part of ethyl acetate was added to soak and wash the filter cake for 3-5 minutes, the filter cake was ground while soaking, then the vacuum was connected, and the ethyl acetate was drawn out; and the vacuum was removed to repeat this operation once again, and then a third part of ethyl acetate was added to wash the filter cake under vacuumizing.

Washing with n-hexane: First the vacuum was removed, 5220 ml of n-hexane (Vₙ₋ₕₑₓₐₙₑ/W_{compound 1}=40.0) was taken and divided into 3 parts evenly. The first part of n-hexane was added to soak and wash the filter cake for 3-5 minutes, the filter cake was ground while soaking, then the vacuum was connected, and the n-hexane was drawn out; then the second part of n-hexane was added to repeat this operation; and then the third part of n-hexane was added to wash the filter cake under vacuum. The filter cake was subjected to suction filtration with a circulating water multipurpose vacuum pump until the product was a powder solid.

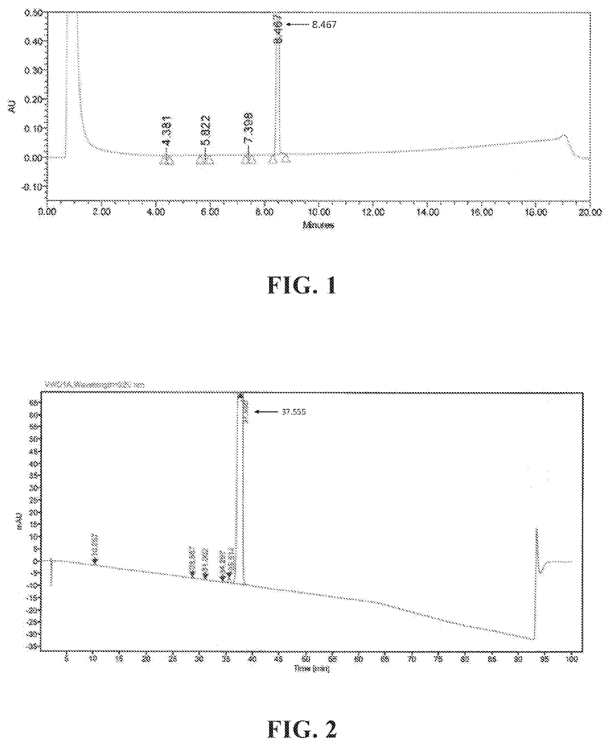

The obtained powder solid was transferred to a 2 L single neck bottle and dried under vacuum at 25-30° C. for at least 16 h. When the weight did not change any more, the drying was stopped, and the obtained powder solid was purified Compound 2 (i.e., purified MC-Val-Cit-PAB-PNP). By testing, the purity reached 99.12%, the maximum single impurity was 0.58%, and the total impurity was 0.88%. The chromatogram is shown in FIG. 1.

### Example 2 The Preparation and Purification for MC-VC-PAB-MMAE

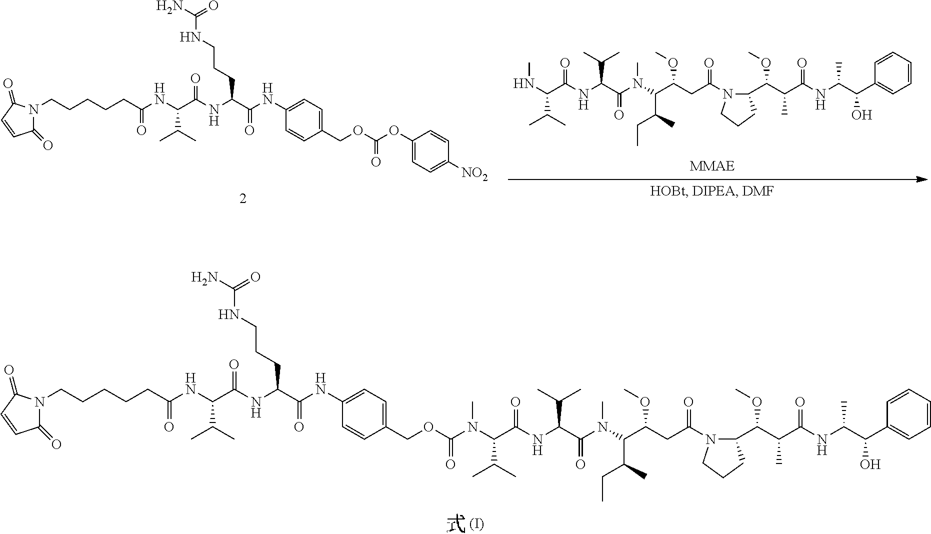

A clean and dry 2 L reaction flask was taken, into which 124.00 g of Compound 2 (168.08 mmol, 1.05 eq), 21.50 g of 1-hydroxybenzotriazole (HOBt) (159.11 mmol) and 460 ml of DMF (V_{DMF}/W_{MMAE}=4.0) were added, stirring was carried out under the protection of nitrogen to dissolve the solid, stirring was started, and the temperature was lowered to 0-5° C. (rotational speed: 100-300 rpm).

When the internal temperature of the above system lowered to the range of 0-5° C., a solution of 114.96 g of MMAE (160.12 mmol) in 460 ml of DMF (VDMF/WMMAE=4.0) was added.

The internal temperature of the reaction system was kept in the range of 0-5° C., and then 20.71 g of N,N-Diisopropylethylamine (160.23 mmol) and 273.03 g of pyridine (3451.71 mmol) were successively added, and the temperature was raised after adding.

The temperature was raised to 25° C., the timing reaction was started, the internal temperature was controlled to 25-30° C., samples were taken for in-process control after reaction for 18 h, and then samples were taken for in-process control every 1 h. When the residual of MMAE was 3.0%, the reaction was stopped.

The above reaction solution was subjected to suction filtration with a circulating water multipurpose vacuum pump, the reaction solution was measured with a measuring cylinder by volume and transferred to a 30 L stainless steel barrel, then a Buchner funnel and a filter flask were washed with 115 ml of DMF (V_{DMF}/W_{MMAE}=1.0), the solution in the filter flask was measured by volume again and transferred to a 30 L stainless steel barrel, and the total volume of the two measurements was V_{reaction solution}. 6363 ml of ethyl acetate about 4 times the volume of V_{reaction solution} was added at one time under stirring (100-300 rpm), and then 12600 ml of n-hexane about 8 times the volume of V_{reaction solution} was dropwise added within 30±10 minutes. Stirring was carried out for about 5 minutes again after dropwise adding and filter suction was carried out to obtain the filter cake which was the unpurified compound of Formula (I).

Next, 3160 ml of ethyl acetate twice the volume of V_{reaction solution} was taken and divided into two parts evenly. First the vacuum was removed, one part of ethyl acetate was taken and added into a Buchner funnel to soak and wash the filter cake for 3-5 minutes, the filter cake was ground while soaking, the vacuum was connected, filter suction was carried out, and this operation was repeated once.

The vacuum was removed, 3160 ml of n-hexane twice the volume of V_{reaction solution} was taken and divided into two parts evenly, one part of n-hexane was taken and added to a funnel to soak and wash the filter cake for 3-5 minutes, the filter cake was ground while soaking, the vacuum was connected, filter suction was carried out, this operation was repeated once, and the solvent was drawn out after the last washing; the filter cake was subjected to suction filtration with a circulating water multipurpose vacuum pump until the product was a powder solid. The obtained solid powder was transferred to a 2 L single neck bottle (the bottle was weighed first) and dried under vacuum at room temperature (18-26° C.) for no less than 5 h until the weight did not change, so as to obtain a dry powder solid.

The above powder solid was dissolved in an appropriate amount of methanol, and was purified by using a preparation and purification system. The specific preparation conditions were as follows: mobile phase A: acetic acid aqueous solution (pH=4.0-5.0), and mobile phase B: acetonitrile. Mobile phase A:B=60:40(V/V), isogradient was used for preparation and purification;

The preparation solution was collected, and the preparation solution obtained was concentrated under reduced pressure at 30±2° C. to a foamed solid. The above foamed solid was dissolved with 1200 ml of methanol (Vₘₑₜₕₐₙₒₗ/W_{MMAE}=10.4), transferred to a 2 L of single neck bottle (the bottle was weighed first), concentrated under reduced pressure at 30-35° C. until the product was a foamed solid, and this operation was repeated twice. The concertation was continued for 0.5 h after a foamed state was formed finally by concentration and there was no liquid drop, and then the obtained solid was vacuum dried with a directly-connected high-speed rotary vane vacuum pump and ground to obtain the purified compound of Formula (I) (i.e., purified MC-VC-PAB-MMAE). The purity was 99.80%, the maximum single impurity was 0.13%, and the total impurity was 0.20%. The chromatogram is shown in FIG. 2.

The present invention has been illustrated by various specific examples. However, a person of ordinary skill in the art can understand that the present invention is not limited to various specific implementations. A person of ordinary skill can make various changes or modifications within the scope of the present invention, and various technical features mentioned in various places in the description can be combined with each other without departing from the spirit and scope of the present invention. Such changes and modifications are within the scope of the present invention.

What is claimed is:

1. A preparation and purification method for an antibody drug conjugate intermediate, the antibody drug conjugate intermediate being a compound, an enantiomer, a racemate or a pharmaceutically acceptable salt thereof as shown in Formula (I), wherein D represents the linked toxin part: 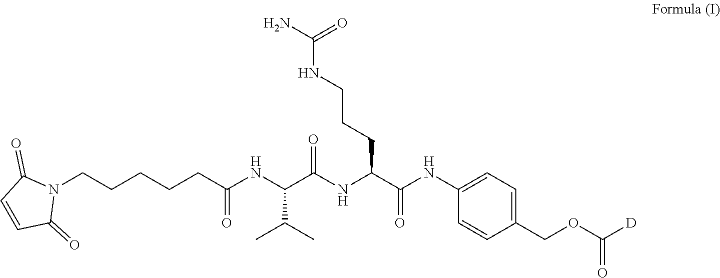 [SMILES: [2H]C(=O)OCc1ccc(NC(=O)[C@H](CCCNC(N)=O)NC(=O)[C@@H](NC(=O)CCCCCN2C(=O)C=CC2=O)C(C)C)cc1]
the synthetic route of the method is as follows: 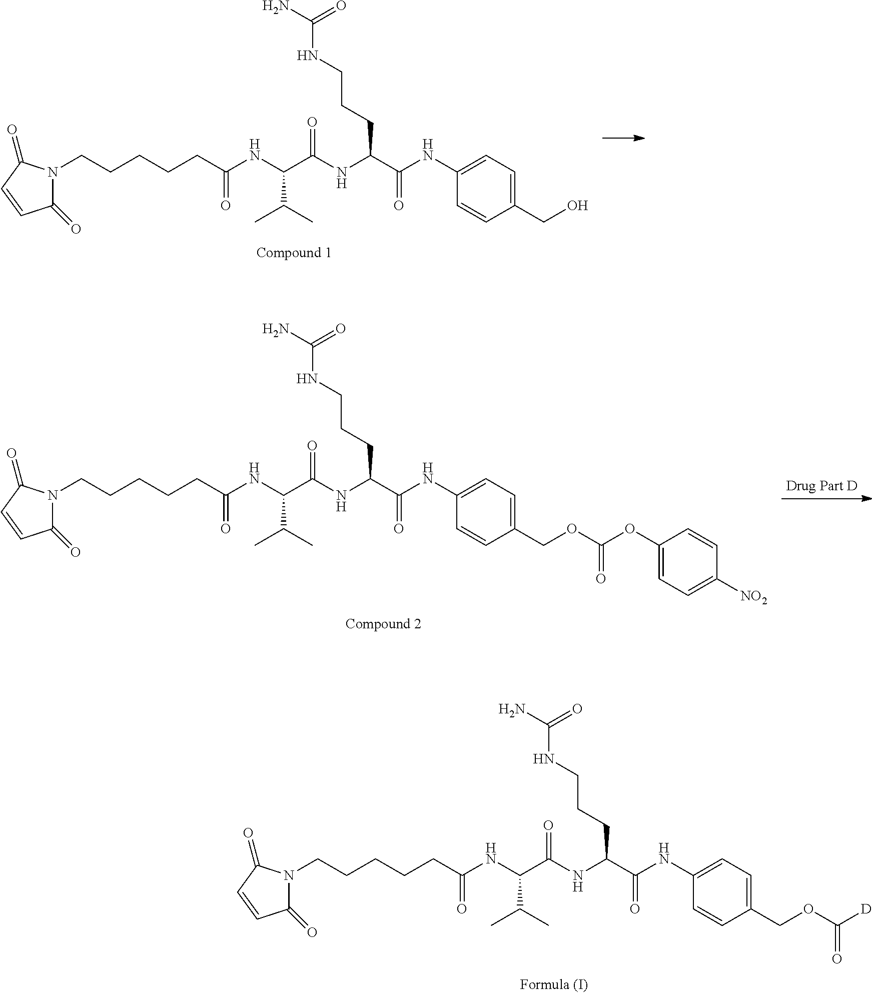 [SMILES: CC(C)[C@H](NC(=O)CCCCCN1C(=O)C=CC1=O)C(=O)N[C@@H](CCCNC(N)=O)C(=O)Nc1ccc(CO)cc1 ; CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1 ; [2H]C(=O)OCc1ccc(NC(=O)[C@H](CCCNC(N)=O)NC(=O)[C@@H](NC(=O)CCCCCN2C(=O)C=CC2=O)C(C)C)cc1]
the toxin part D is an auristatin cytotoxic agent, an Anthramycin cytotoxic agent, an anthracycline cytotoxic agent or a puromycin cytotoxic agent, wherein the auristatin cytotoxic agent comprises MMAE, MMAF, MMAD or derivatives thereof, the Anthramycin cytotoxic agent comprises anthramycin or derivatives thereof; the anthracycline cytotoxic agent comprises daunorubicin, adriamycin, epirubicin, idarubicin, valrubicin, mitoxantrone or derivatives thereof; the puromycin cytotoxic agent comprises puromycin or derivatives thereof;
the method specifically comprises the following steps:
A. dissolving Compound 1 in an appropriate amount of Solvent 1, and successively adding bis(4-nitrobenzene) carbonate and organic base, wherein the number of moles of the added bis(4-nitrobenzene) carbonate and the number of moles of the added organic base are greater than that of Compound 1;
B. obtaining the filtrate by suction filtration after appropriate reaction time;
C. successively adding a sufficient amount of ethyl acetate and n-hexane to the filtrate obtained in Step B, stirring for an appropriate time after n-hexane has been dropwise added, and obtaining the filter cake by suction filtration;
D. successively washing the filter cake obtained in Step C with an appropriate amount of ethyl acetate and n-hexane, and obtaining the filter cake by suction filtration;
E. dissolving the filter cake obtained in Step D in a mixed solution of acetic acid and methanol, adding an appropriate amount of purified water, stirring for an appropriate time after the purified water is added, and obtaining the filter cake by suction filtration;
F. successively washing the filter cake obtained in Step E with an appropriate amount of purified water, methanol, ethyl acetate and n-hexane, and obtaining Compound 2 (MC-Val-Cit-PAB-PNP) after suction filtration and drying;
G. dissolving Compound 2 and a triazole-based compound in an appropriate amount of Solvent 2 to form Solution X, dissolving the conjugated toxin part D in Solvent 3 to form Solution Y, adding Solution Y into Solution X, and mixing evenly to form Solution Z;
H, adding an appropriate amount of organic base to Solution Z to adjust the pH of the system and catalyze the reaction;
I. obtaining the filtrate by suction filtration after appropriate reaction time;
J. successively adding an appropriate amount of ethyl acetate and n-hexane to the filtrate in Step I, stirring for an appropriate time, and obtaining the filter cake by suction filtration;
K. successively washing the filter cake obtained in Step J with ethyl acetate and n-hexane, and obtaining the filter cake by suction filtration;
L. dissolving the filter cake obtained in Step K in an appropriate amount of methanol solution, preparing and purifying by preparative liquid chromatography, and collecting a preparation solution;
M. concentrating the preparation solution obtained in Step L under reduced pressure;
N dissolving the concentrate obtained under reduced pressure in Step M with an appropriate amount of methanol, and then concentrating under reduced pressure again;
O. vacuum drying the concentrate obtained under reduced pressure in Step N to obtain the purified compound as shown in Formula (i);
wherein:
Solvent 1 in Step A, Solvent 2 and Solvent 3 in Step G are polar solvents; preferably, Solvent 1, Solvent 2 and Solvent 3 are each independently selected from one or more of DMF, DMA and NMP; and more preferably, Solvent 1, Solvent 2 and Solvent 3 are DMF.

2. The method as described in claim 1, wherein the molar ratio of Compound 1 in Step A to bis(4-nitrobenzene) carbonate is about 1:1.8, and the molar ratio of Compound 1 to organic base 1 is about 1-1.2.

3. The method as described in claim 2, wherein the molar ratio of Compound 1 in Step A to bis(4-nitrobenzene) carbonate is 1:1.5-2, and the molar ratio of Compound 1 to organic base 1 is 1:1-1.5.

4. The method as described in claim 2, wherein the weight volume ratio (g/ml) of Compound 1 to ethyl acetate in Step C is about 1:30.0, and the weight volume ratio (g/ml) of Compound 1 to n-hexane in Step C is about 1:60.0.

5. The method as described in claim 4, wherein the weight volume ratio (g/ml) of Compound 1 to ethyl acetate in Step C is 1:25-35, and the weight volume ratio (g/ml) of Compound 1 to n-hexane m Step C is 1:55-65.

6. The method as described in claim 4, wherein the weight volume ratio (g/ml) of Compound 1 to acetic acid in Step E is about 1:7.0, the weight volume ratio (g/ml) of Compound 1 to methanol in Step E is about 1:1.0, and the weight volume ratio (g/ml) of Compound 1 to purified water in Step E is about 1:20.0.

7. The method as described in claim 6, wherein the weight volume ratio (g/ml) of Compound 1 to acetic acid in Step E is 1:6-8, the weight volume ratio (g/ml) of Compound 1 to methanol in Step E is 1:0.5-1.5, and the weight volume ratio (g/ml) of Compound 1 to purified water in Step E is 1:15-25.

8. The method as described in claim 6, wherein the molar ratio of Compound 2 to triazole-based compound in Step G is about 1:1, and the molar ratio of Compound 2 to toxin part D is about 1:1.

9. The method as described in claim 8, wherein the molar ratio of Compound 2 to triazole-based compound in Step G is 1:0.8-1.2, and the molar ratio of Compound 2 to toxin part D is 1:0.8-1.2.

10. The method as described in claim 8, wherein the organic base in Step A and the organic base in Step H are each independently selected from one or more of N,N-Diisopropylethylamine, triethylamine and pyridine.

11. The method as described in claim 10, wherein the organic base in Step A is N,N-Diisopropylethylamine, and two types of organic bases, N,N-Diisopropylethylamine and pyridine, are added in Step H.

12. The method as described in claim 11, wherein the molar ratio of Compound 2 to N,N-Diisopropylethylamine added in Step H is about 1:1, and the molar ratio of Compound 2 in Step G to pyridine added in Step H is about 1:20.5.

13. The method as described in claim 12, wherein the molar ratio of Compound 2 in Step G to N,N-Diisopropylethylamine added in Step H is 1:0.8-1.2, and the molar ratio of Compound 2 in Step G to pyridine added in Step H is 1:19-25.

14. The method as described in claim 11, wherein the volume of the ethyl acetate added in Step J is 3.5-4.5 times the volume of the filtrate, and the volume of the n-hexane added is 7-9 times the volume of the filtrate.

15. The method as described in claim 11, wherein the preparation conditions of high performance liquid chromatography in Step L are as follows: mobile phase A is an acetic acid aqueous solution with pH=4.0-5.0, mobile phase B is acetonitrile, mobile phase A:B=60:40(V/V), isogradient is used for preparation and purification.

16. The preparation and purification method as described in claim 1, wherein the structure of the antibody drug conjugate intermediate is as shown in Formula (1-11):

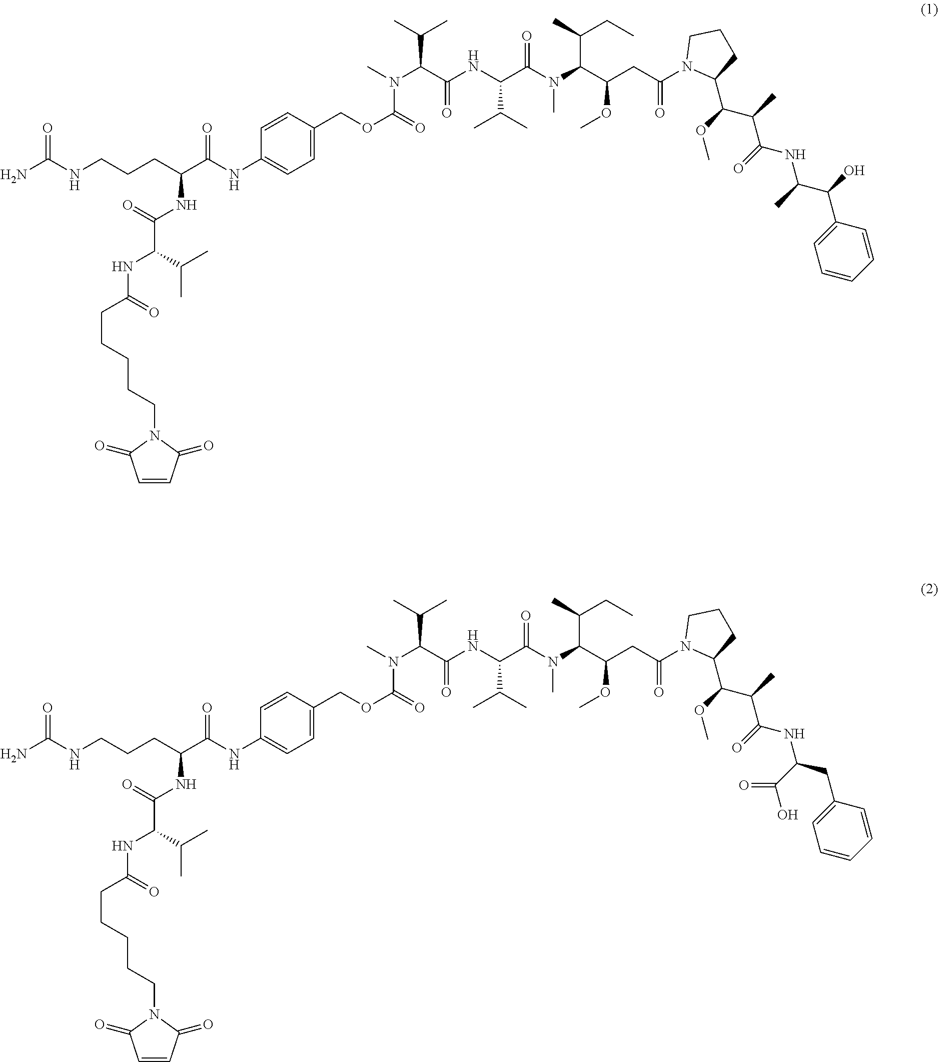 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C ; CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C]

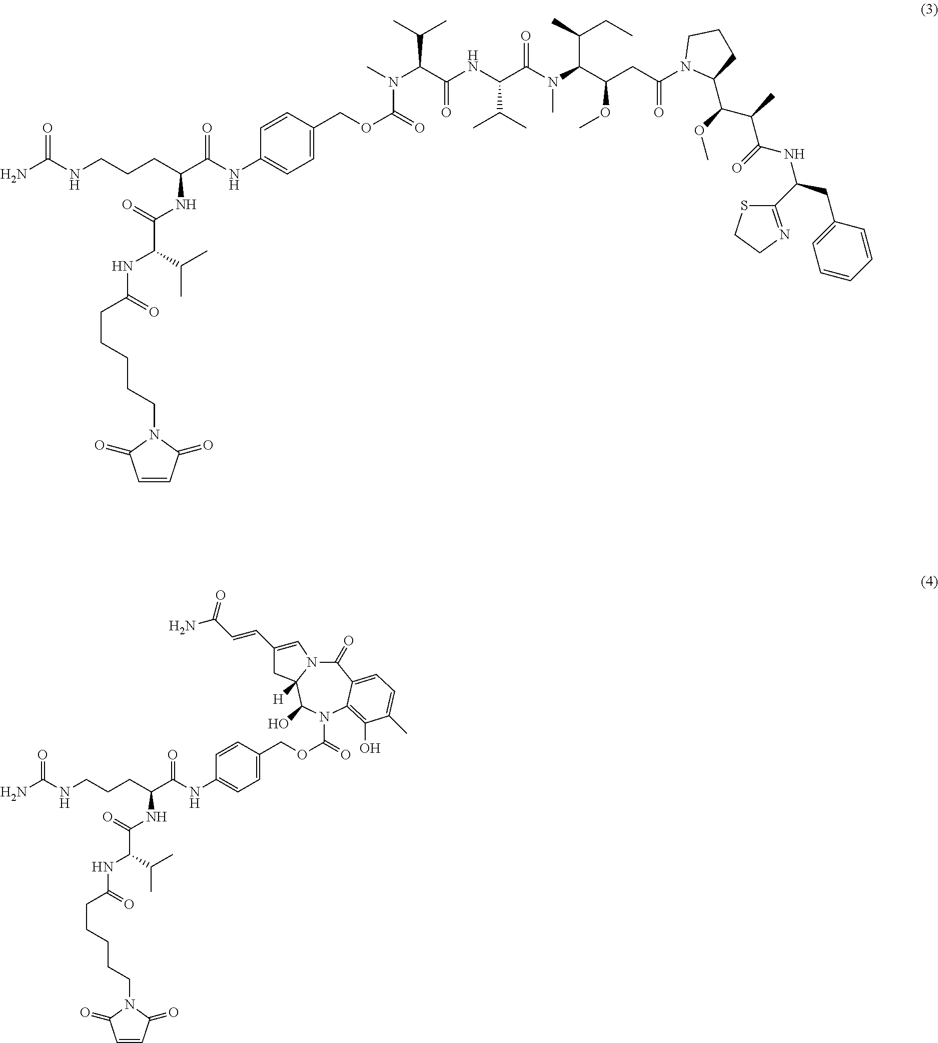 [SMILES: CC[C@H](C)[C@@H]([C@@H](CC(=O)N1CCC[C@H]1[C@H](OC)[C@@H](C)C(=O)N[C@@H](Cc1ccccc1)C1=NCCS1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C ; Cc1ccc2c(c1O)N(C(=O)OCc1ccc(NC(=O)[C@H](CCCNC(N)=O)NC(=O)[C@@H](NC(=O)CCCCCN3C(=O)C=CC3=O)C(C)C)cc1)[C@@H](O)[C@@H]1CC(/C=C/C(N)=O)=CN1C2=O]

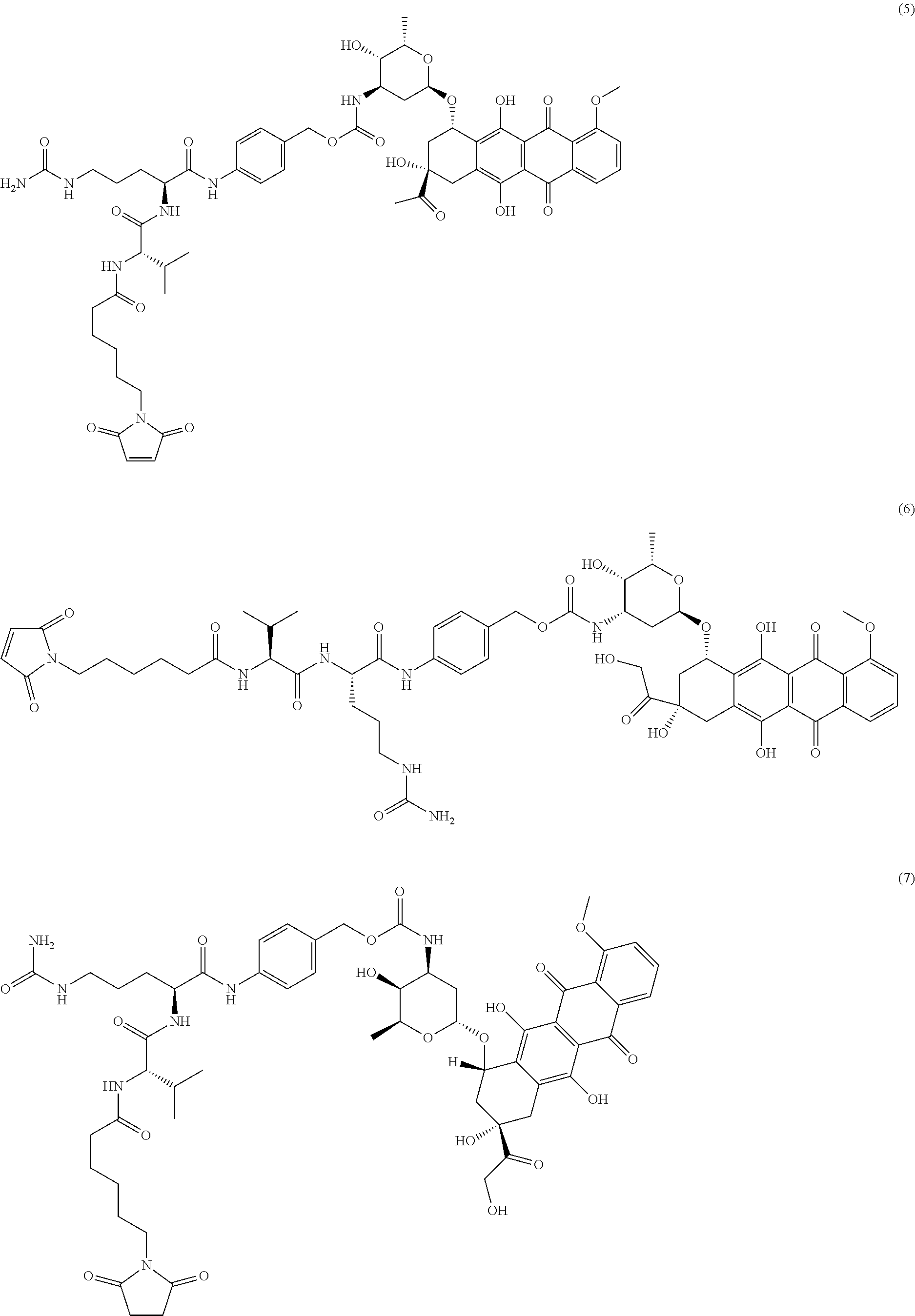

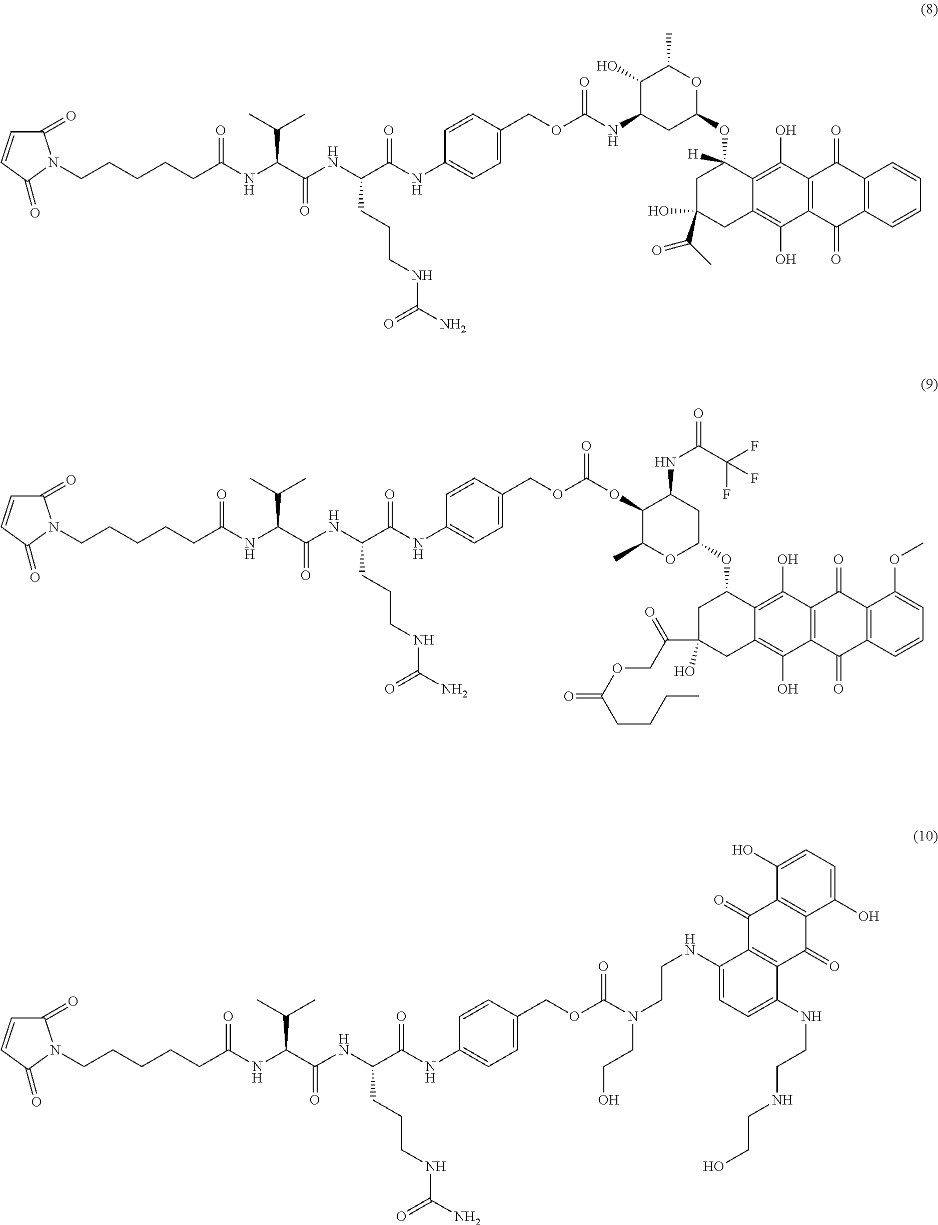

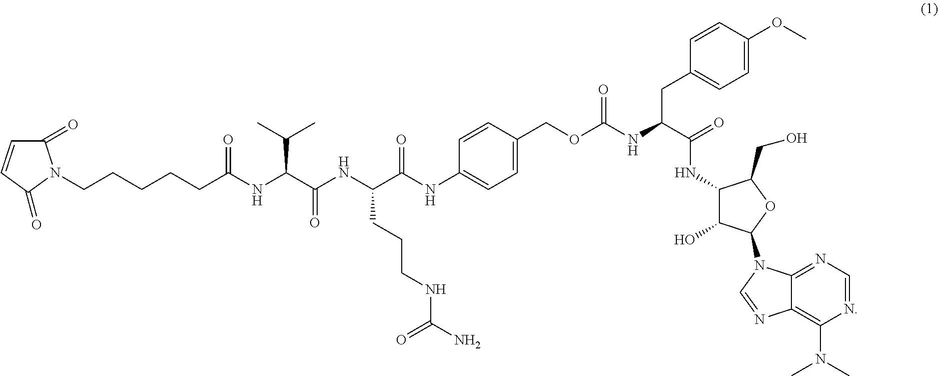 [SMILES: COc1ccc(C[C@H](NC(=O)OCc2ccc(NC(=O)[C@H](CCCNC(N)=O)NC(=O)[C@@H](NC(=O)CCCCCN3C(=O)C=CC3=O)C(C)C)cc2)C(=O)N[C@H]2[C@@H](O)[C@H](n3cnc4c(N(C)C)ncnc43)O[C@@H]2CO)cc1]

17. The preparation and purification method as described in claim 1, wherein the temperature in Step A is controlled within the range of −5-5° C.

18. The preparation and purification method as described in claim 1, wherein the temperature in Step B is controlled within the range of 25-30° C.

19. The preparation and purification method as described in claim 1, wherein Step D can be repeated 1-5 times.

20. The preparation and purification method as described in claim 1, wherein the washing times in Step F are 1-5 times.

21. The preparation and purification method as described in claim 1, wherein the drying temperature in Step F is 25-30° C.

22. The preparation and purification method as described in claim 1, wherein the triazole-based compound in Step G are one or more of 1-hydroxybenzotriazole, 1-hydroxy-7-azobenzotriazole and ethyl 1-hydroxyl-1H-1,2,3-triazole-4-carboxylate, preferably 1-hydroxybenzotriazole.

23. The preparation and purification method as described in claim 1, wherein the temperature in Step G is controlled within the range of −5-5° C.

24. The preparation and purification method as described in claim 1, wherein the temperature in Step H is controlled within the range of −5-5° C.

25. The preparation and purification method as described in claim 1, wherein the reaction temperature in Step I is 25-30° C.

26. The preparation and purification method as described in claim 1, wherein the washing times in Step K are 1-5 times.

27. The preparation and purification method as described in claim 1, wherein the temperature for concentration under reduced pressure in Step M is 25-35° C.

28. The preparation and purification method as described in claim 1, wherein Step M is to concentrate the preparation solution obtained in Step L under reduced pressure to a foamed solid state.

29. The preparation and purification method as described in claim 1, wherein the temperature for concentration under reduced pressure in Step N is 25-35° C.

30. The preparation and purification method as described in claim 1, wherein Step N is to dissolve the concentrate obtained under reduced pressure in Step M with an appropriate amount of methanol, and then concentrate under reduced pressure again to a foamed solid state.

31. The preparation and purification method as described in claim 1, wherein Step N can be repeated 1-5 times.

32. The preparation and purification method as described in claim 1, wherein Step A, Step B, Step G, Step H and Step I are all carried out under the protection of nitrogen.
